(19) United States
(12) Patent Application Publication
(10) Pub. No.: US 2026/0327694 A1
(43) Pub. Date: Oct. 22, 2026

(54) Combined Cell-Specific Labelling and Enrichment of Biomarkers

(71) Applicant: Robert Bosch GmbH, Stuttgart (DE)

(72) Inventors: Anke Timm, Renningen (DE); Samir Kadic, Renningen (DE); Christian Grumaz, Waiblingen (DE); Sebastian Pilsl, Stuttgart (DE); Michael Knapp, Wiernsheim (DE); Astrid Lux, Weil Der Stadt (DE)

(21) Appl. No.: 19/122,484

(22) PCT Filed: Sep. 29, 2023
(86) PCT No.: PCT/EP2023/077002

§ 371 Date: Apr. 18, 2025

(30) Foreign Application Priority Data
Oct. 19, 2022 (DE) 10 2022 211 089.7

(51) Int. Cl. C12Q 1/6858; C12Q 1/686

(52) U.S. Cl. CPC C12Q 1/6858; C12Q 1/686

(57) ABSTRACT

Please add the following paragraph on a new page after claim 17 set forth on page 31 of the Applicant's specification.

A microfluidic method is for the combined cell-specific labeling and enrichment of biomarkers in a reaction compartment. The method includes providing a microfluidic device having at least one carrier substrate for receiving a sample liquid. The carrier substrate has at least one microcavity, and at least one electrode arranged at or in the microcavity to generate an electrical field configured to trap a cell and/or a primer bead in the microcavity. The method also includes loading the at least one microcavity of the microfluidic device with a cell and trapping it, and loading the at least one microcavity of the microfluidic device with a primer bead having at least one primer population coupled thereto and trapping the primer bead.

The present invention relates to a microfluidic method for combined cell-specific labeling and enrichment of biomarkers, as well as to a control unit configured to execute and/or control the steps of the method, according to the genus of the independent claims. The subject matter of the present invention is also a computer program.

### PRIOR ART

A biomarker is understood to mean a measurable biological feature with prognostic or diagnostic validity. In molecular diagnostics, diseases based on nucleic acid biomarkers can be detected and appropriate therapies can be initiated. These biomarkers may be deoxyribonucleic acid (DNA) or ribonucleic acid (RNA) biomarkers.

In this context, the query of targeted, multiple biomarkers is of high value and is used, for example, in infection diagnostics, in which different species are queried simultaneously, or in tumor diagnostics, in which different mutation sites in the genome are simultaneously queried. The number of these so-called biomarker targets varies from a few dozen to tens of thousands depending on the assay. The goal is always to specifically enrich these biomarker targets against the non-informative but usually highly concentrated background, in order to make the readout of the biomarker targets easier and more robust, for example by means of a real-time quantitative polymerase chain reaction (qPCR), digital droplet polymerase chain reaction (ddPCR), or parallel sequencing (next-generation sequencing, NGS).

For the enrichment of nucleic acids, two fundamentally different principles are primarily pursued-on the one hand a multiplex PCR with specific primers or primer pairs and, on the other hand, a hybridization-based positive selection with specific probes. However, what these enrichment methods have in common is that they involve a number of successive process steps and may take several hours to a day to complete. This makes them unsuitable for use at the point-of-care in a lab-on-chip environment. In addition, several nanograms of the nucleic acids are required as the starting material, which requires additional pre-amplification, for example in case of low-input sample material (whole genome amplification (WGA)) and/or whole transcriptome amplification (WTA) which in turn costs additional process steps and time.

In numerous disciplines of biology, such as immunology, neurobiology, oncology or stem cell and developmental biology, it is of interest to analyze as many (several hundred thousand to millions) nucleated cells of a heterogeneous cell population as possible in the shortest possible time (hours to a few days) at the single cell level. Single cell analysis can be performed either molecularly at the DNA, RNA, and/or protein levels, for example, as well as functionally in the form of cell cultivation or/and drug testing.

Droplet-based, micro-trough-based or valve-based microstructured substrates are known with which cells can be spatially isolated. However, none of these systems meets the requirements to perform highly parallelized enrichment of DNA and/or RNA-encoded biomarkers with a clear molecular labeling of the target amplifiers, for example for point-of-care analyses, in few process steps.

### DISCLOSURE OF THE INVENTION

According to the present invention, there is provided a microfluidic method for combined cell-specific labeling and enrichment of biomarkers in a reaction compartment with the features of the independent claim.

A device with which the method according to the invention can be performed, as well as a method for operating the same, is described in the yet unpublished German application with file number 102022203848.7 with the filing date on 19.04.2022 and priority date on 20.04.2022. The content of this application is included in the description by reference to this application. It should be noted that it is undoubtedly discernible that the features of this application contribute to solving the technical problem underlying the present invention.

The microfluidic method for combined cell-specific labeling and enrichment of biomarkers in a reaction compartment comprises at least steps a)-g).

In step a), a microfluidic device is provided as described in the yet unpublished application with file number 102022203848.7. This comprises at least one carrier substrate for receiving a sample liquid. The carrier substrate comprises at least one microcavity and further comprises at least one electrode arranged at or in the microcavity in order to generate an electrical field configured to trap a cell, in particular a nucleated cell, and/or a primer bead in the microcavity.

This microfluidic device can, for example, integrate around 30,000 individually actuatable microcavities or reaction compartments per square centimeter. For example, these microcavities have a square base area and a cavity width of around 50 μm. For example, the microcavities are arranged in a regular matrix. The microcavities may trap or repel individual cells and/or particles. Individual cells may sediment into a microcavity in a controlled manner. In addition, for example, irrelevant cells may be removed from the microcavities, whereas relevant cells may be retained by using an electric field to generate a dielectrophoresis cage (DEP cage) in the microcavity, which may be opened, closed or switched off depending on the current strength, for example.

The DEP cages can be realized, for example, by active components, for example as transistors or storage elements. These are integrated within individual electrodes in complementary metal-oxide semiconductor (CMOS) technology.

In step b), the at least one microcavity of the microfluidic device is loaded with a cell, in particular a nucleated cell, and the cell is trapped in the microcavity. To this end, the DEP cage is closed by a voltage change. Further cells outside the DEP cage can no longer enter the same while the cell within the DEP cage is stably trapped. In this way, it is ensured that only a single cell is located within the microcavity.

In step c), the at least one microcavity of the microfluidic device is loaded with a primer bead and the primer bead is trapped in the microcavity. For example, the primer bead comprises at least one primer population coupled thereto. Once the primer bead is sedimented into the microcavity filled with the cell, the DEP cage is closed by a voltage change. Further primer beads outside the DEP cage can no longer enter the same while the cell and primer beads within the DEP cage are stably trapped. In this way, it is ensured that only a single primer bead is located within the microcavity.

Steps b), c) and d) may also be performed in any reversed order, in particular steps b) and c) may be performed in reverse order.

In step d), an amplification mixture is introduced into the at least one microcavity. For this purpose, the constituents located in the amplification mixture diffuse into at least one microcavity.

The amplification mixture comprises a buffer compatible with its constituents, a polymerase, in particular an SD polymerase, such as Bst, Bst2.0, Bst3.0, EquiPhi, Phi29, Vent exo- and/or others. Furthermore, the amplification mixture optionally comprises a reverse transcriptase (RT), e.g., RTx, SSIV, and/or others. In addition, the amplification mixture comprises deoxyribonucleoside triphosphates (dNTPs) and/or analogs thereof. The dNTPs may additionally be modified. In addition, the amplification mixture may also include constituents that positively affect the amplification reaction and/or cell lysis and/or primer release, for example. Such constituents include, for example, additives such as polyethylene glycol (PEG), dithiothreitol (DTT), detergents, or also enzymes such as restriction enzymes or single strand break-inducing enzymes (nicking enzymes).

In step e), the amplification mixture is displaced above the at least one microcavity by a non-aqueous phase, in particular by an oil phase or air, such that the microcavity is closed in the area of its opening by the non-aqueous phase and thus represents a closed reaction compartment. If the microfluidic device comprises a plurality of microcavities, then all microcavities represent isolated and closed reaction compartments, each containing a cell and a primer bead with the amplification mixture.

In step f), conditions are induced which lead to lysis of the cell and release of the primers from the primer bead. Such conditions are produced, for example, by raising the temperature, for example to above 70 °C for more than one minute, and/or by electroporation and/or by ultrasound, and/or by other conditions or reagents conducive to this reaction which have been introduced, for example, by means of the amplification mixture.

In step g), the at least one primer population binds to biomarker regions of the DNA and/or RNA which were released from the cell during cell lysis, so that amplification of the biomarker target region takes place and the biomarker target amplifier is labeled in a cell-specific manner.

For the amplification, DNA double strands and/or secondary and/or tertiary structures of the RNA are denatured, so that the DNA and/or RNA are present as single strands, for example by means of suitable temperatures. In a further step, the primers attach to the DNA and/or RNA, thereby initiating amplification of the corresponding DNA and/or RNA.

Advantageously, the method according to the invention is an automated method suitable for a lab-on-chip with which DNA and/or RNA-encoded biomarkers can be enriched in a highly parallelized manner and the biomarker target amplification can be uniquely assigned to its original cell via molecular cell bar code. The target amplifiers are thus available for downstream detection methods, such as for NGS analysis, within a few minutes to hours, making the method suitable for point-of-care analysis.

No further equipment is necessary for this.

A great advantage is that only a few process steps are needed. For example, a previous pre-amplification/WGA/WTA or previous pooling of several pre-sorted single cells is omitted. This leads to a significant reduction in the process time, reagents, costs and steps required.

The method ensures a unique labeling of each individual cell, and thus nearly 100% pairing efficiency, with an equally nearly 100% loading efficiency. This means that each microcavity can be loaded accurately with one cell and accurately with a primer bead. In addition, several process steps may be performed in succession, as the microcavities can be loaded and unloaded several times.

Simultaneous enrichment of the relevant DNA and/or RNA-biomarker target regions may occur at a degree of parallelization of, for example, 30,000 microcavities per cm².

It is further advantageous that the sample input volume is theoretically unlimited and different samples can be entered, for example blood or saliva.

In addition, the method only allows downstream analysis of cells that are, for example, relevant.

Furthermore, it is advantageous that the primer beads entering the microcavity are trapped in the microcavity together with the cell and that no further cells or primer beads can enter the microcavity.

Further advantageous embodiments result from the dependent claims.

In one advantageous embodiment, different populations of primers are reversibly coupled to the primer beads. For example, the primer bead comprises a polystyrene, particularly in the form of a polystyrene bead. The various primer populations are in particular biomarker-specific forward primers (SFP), biomarker-specific reverse primers (SRP) and/or random primers (RP).

The primer populations are coupled to the primer beads, for example, via base pairings that can be released by certain temperatures or via specific sequences or other chemical modifications in the base composition that can be released, for example, by enzymes or light of a certain wavelength. This may be, for example, an interface for a restriction enzyme or the base analog dUTP for uracil DNA glycosylase (UDG).

In a further advantageous embodiment, the SFP and SRP each comprise a selection marker coupled to a universal sequence, a unique cell bar code sequence, and corresponding specific target sequences flanking the biomarker target regions in forward and reverse orientation.

Each primer bead clonally contains a unique cell bar code sequence (BC₁₋ₙ) with a length of >8 bases. The selection marker is, for example, biotin at the 5′ end of the SFP or SRP.

In a particularly advantageous embodiment, the selection marker is coupled to an internal, for example modified, base of the universal sequence, wherein the universal sequence is extended towards the 5′ end by a single-strand break sequence (nicking site), which induces a single-strand break via a corresponding nicking enzyme.

By means of the selection marker, subsequent positive selection of the biomarker target amplification is possible. For example, the universal sequence is needed for subsequent amplification of a sequencing library or known sequence having a function of choice, for example a restriction interface, in particular for the release of the biomarker target amplifier, for example from a capture molecule.

It is also advantageous that the labeling of the individual biomarker target amplifiers with a cell bar code allows them to be clearly assigned to the individual cells. Further analyses of a plurality of cells, for example molecular genetic analyses using NGS, in which relevant biomarkers can be assigned to individual cells, for example from isolated cells of tissue biopsies, can be of great benefit, for example in diagnostics and therapy monitoring of tumor patients.

As the cell and primer beads are trapped in the microcavity and no further cells or primer beads can enter the microcavity, unique single cell labeling is possible as each primer bead is provided with a unique molecular cell bar code.

It is contemplated in a further advantageous embodiment to employ multiple, in particular 2-4, SFP and SRP with different specific forward and reverse target sequences flanking the biomarker target region for each biomarker target region. In this case, for example, they can also be used at different concentrations.

Advantageous here is that, for example, with an increasing number of SFP or SRP per biomarker target region, the chances of the SFP or SRP binding to the target region increase in addition to the amplification rate or, in another example, a more uniform amplification is achieved in comparison to other biomarker target regions by adjusting the concentration of the SFP or SRP of individual biomarker target regions that are, for example, more difficult to amplify.

Furthermore, in one embodiment, it is advantageous if SFP or SRP further comprise a unique molecular identifier (UMI) in the form of a randomized sequence. For example, the UMI can be used between the universal sequence and the cell bar code sequence, or between the cell bar code sequence and the forward or reverse target sequence. The UMI in particular has a length of >6 bases.

In one advantageous embodiment, the primer beads are designed so that the SFP and SRP are already reversibly bonded to the primer beads via the selection marker. In the case of a reversible compound, the SFP and SRP are, for example, via antibodies, terminal chemical modifications, such as a biotin coupling. Further alternatively, the SFP and SRP may also be reversibly bonded to the primer beads via aptamers, temperature stable groups or pH sensitive groups or light-splittable groups such as o-nitrobenzyl.

In this embodiment, the random primers are supplied with the amplification mixture for amplification, in particular for an ssMDA (described below).

Advantageously, the target amplifiers are produced directly on the primer bead and can be held (trapped) after removal of the non-aqueous phase by means of the DEP forces and purified directly in the microcavity. Thus, in this embodiment, no capture molecules are necessary because the amplification of the biomarker targeting region is done via the SFP and SRP attached to the primer beads.

A selection marker can be omitted here.

A further advantage is that the biomarker target amplifiers immobilized on the primer beads held in the DEP cage may be further processed in the microcavity as needed, for example by adding further primer beads or liquid reagents.

In a particularly advantageous embodiment, the universal sequence of the SFP and SRP is selected so that a direct attachment point in the form of a specific sequence is introduced for further amplification, in particular for the completion of a sequencing library.

Advantageous here is that an additional duplication or preparation of the purified biomarker target amplifiers is possible using other sequencing technologies, for example another manufacturer.

For example, the asymmetric design required for this can be realized in such a way that all of the SFPs receive a first universal sequence and all SRPs receive a second universal sequence that differs from the first universal sequence. In this way, only a highly standardized PCR, for example an index PCR, is needed before the sequencing library can be sequenced.

Flexible use of the universal sequences ensures that the method is compatible with downstream, preferably applied analysis methods, such as various NGS technologies, for single cell analysis.

In one advantageous embodiment, the amplification mixture is cell lysis-supporting.

This may be achieved, for example, by a reagent that lyses cells and releases nucleic acids but does not inhibit or even promote the subsequent reaction, for example, with a detergent or by a change in osmolarity.

Advantageous here is that no individual process steps are necessary for the cell lysis and mixture with the amplification mixture and the respective reagents can also be pre-mixed in advance. This leads to savings in time, reagents and costs. Furthermore, the reduced steps minimizes dilution effects and losses that would otherwise result from repeated rinsing. This results in an increase in efficiency.

In an alternative or additional embodiment, cell lysis occurs after the introduction of the amplification mixture by brief heating, for example to >50 °C for >1 min. In a further alternative or additional embodiment, cell lysis occurs by increasing the pressure within the microcavities, for example by applying positive pressure.

In a particularly advantageous embodiment, the amplification is an isothermal amplification, in particular a semi-specific multiple displacement amplification (ssMDA). This method is also described in the yet unpublished German application with file number 102022211087.0.

The ssMDA offers the option of simultaneously enriching DNA and RNA from very small quantities, both whole genomically and whole transcriptomically as well as preferentially amplifying and enriching individual biomarker-relevant areas, i.e., the biomarker target regions.

This is achieved by using specific forward primers and reverse primers for amplification of the biomarker target regions in addition to the random primers, for example RP6 with the sequence NNNNNN or RP9 with the sequence NNNNNNNNN, wherein N stands for one of the bases A, C, T, or G in ssMDA. With regard to the biomarker target region, these attach flanking forward up to 3 kilobases (kb) upstream to the DNA or RNA and flanking reverse up to 3kb downstream. The SFP and SRP are additionally equipped with a selection marker, for example biotin.

A downstream purification of the reaction, for example with a matching capture molecule, such as streptavidin, therefore allows for a direct enrichment of the specific biomarker target amplifiers compared to the background amplifier created in parallel.

Particularly preferably, the SFP and SRP have a single-strand break interface in the ssMDA. This single-strand break interface provides an additional amplification start point for a polymerase upon generation of the single-strand break through a suitably added single-strand break-inducing endonuclease (nicking enzyme). As a result, the biomarker target amplification at these locations may be continued without further primer hybridizations, which increases processivity.

During amplification, a single strand break-inducing endonuclease is added to the polymerase for this purpose. The endonuclease is preferably selected from the group consisting of Nt. AlwI, Nb. BbvCI, Nt. BbvCI, Nb. BsmI, Nt. BsmAI, Nt. BspQI, Nb. BsrDI, Nb. BssSI, Nt. BstNBI, Nb. BtsI and Nt. CviPII.

In ssMDA, the RP are preferably primers which have at least one modification selected from the group consisting of LNA (Locked Nucleic Acid), MGB (Minor Groove Binder), C-5 propynyl-deoxycytidine, C-5 propynyl-deoxyuridine, aminoethyl-phenoxazine-deoxycytidine, 5-methyl-deoxycytidine, 2-amino-deoxyadenosine, trimethoxystilbene, pyrene and spermine. ZNA primers (Zip Nucleic Acids) are particularly preferred. These are spermine-modified primers.

These have the advantage that they form particularly temperature stable hybridized nucleic acids with single strand nucleic acids (DNA and/or RNA) and that higher hybridization temperatures can be used without the need for sequence extension of the primers. They also reduce the self-hybridization of the primers if the primers are loaded with spermine. This is particularly advantageous if the primers used are short and the isothermal amplification is performed at temperatures of at least 45 °C.

The ssMDA runs isothermally, for example at temperatures between 40-72 °C for more than 10 minutes.

Preferably, an SD polymerase is used for the ssMDA.

For example, the SD polymerase may be an enzyme mutant of the DNA polymerase of the Bacillus subtilis phage Phi 29, in particular EquiPhi 29, or the Thermococcus litoralis, in particular Vent (exo-). The SD polymerase vent (exo-) has the advantage that it is stable and processive up to temperatures of 100 °C. Therefore, it may already be added before denaturing the DNA and/or RNA without being damaged by the high denaturation temperature. However, these SD polymerases do not have RNA processivity.

In this respect, the SD polymerase is preferably an enzyme mutant of the DNA polymerase of the Bacillus stearothermophilus.In addition to their processivity towards DNA, these SD polymerases also exhibit processivity towards RNA. Particularly preferred enzyme mutants of the DNA polymerase of the Bacillus stearothermophilus are selected from the group consisting of Bst, Bst 2.0 and Bst 3.0. Among these enzyme mutants, Bst 3.0 has the highest RNA processivity, which is why Bst 3.0 is particularly preferred.

The SD polymerase is a DNA polymerase suitable for isothermal amplification reactions and is already known for high strand displacement activity in amplification reactions.

The use of SD polymerase has several advantages. It allows isothermal amplification. In addition, some SD polymerases tolerate high temperatures during isothermal amplification, which increase the reaction rate to such an extent that a significant reduction in reaction duration is possible. Finally, some SD polymerases may use RNA as a template for amplification in addition to DNA.

Although the SD polymerase is capable of processing RNA in addition to DNA, it is preferred that at least one reverse transcriptase is additionally used in the ssMDA. In particular, this is RTx or SSIV. In principle, however, all reverse transcriptases that process under similar reaction conditions are conceivable, as is the SD polymerase used. This specifically supports the amplification of RNA so that the required amplification period can be further reduced. If an SD polymerase that does not have RNA processivity is used, then the use of reverse transcriptase is necessary to process RNA in addition to DNA.

For amplification using ssMDA, the DNA and/or RNA to be amplified is denatured, in particular by heating it to a suitable temperature. In the case of thermophilic polymerases, for example vent (exo-) polymerase, denaturation preferably occurs at a temperature in the range of 75 °C to 98 °C, and more preferably at a temperature in the range of 80 °C to 95 °C. Preferably, the heating is performed for a period of time of more than 10 seconds, more preferably for a period of time in the range of 30 seconds to 120 seconds.

In the case of non-thermophilic SD polymerases, for example Bst 3.0, temperatures of 65-85 °C, and more preferably temperatures of 72-80 °C, are preferably used.

The primers for hybridizing the single-stranded DNA and/or RNA may preferably already be brought together with the DNA and/or RNA prior to denaturation, more preferably by being already released in the amplification mixture. The hybridization of the primers to the DNA and/or RNA single strands is then preferably performed at a temperature in the range of 4 °C to 65 °C for a period of time, preferably greater than 10 seconds.

The ssMDA is preferably carried out at a constant temperature with a value in the range of 40 °C to 72 °C. A preferred amplification time is in the range of 10 minutes to 120 minutes. The higher the temperature during amplification, the faster it takes place and the shorter less the amplification period can usually be advantageously chosen.

The advantage of ssMDA is that DNA and RNA biomarkers can be simultaneously amplified and enriched automatically in the same reaction compartment from single cells, which represents a huge improvement and simplification of the biomarker enrichment, also in terms of the effort required, the duration of the experimental implementation, the number of work steps and the associated costs.

In one embodiment alternative to the ssMDA, amplification is performed by means of reverse transcriptase PCR. Here, for example, only one SFP and SRP are employed per biomarker target region. For example, in this embodiment, the amplification mixture comprises a Taq polymerase and a reverse transcriptase.

Furthermore, in one embodiment, it is advantageous if the non-aqueous phase above the microcavities, which had closed them in the area of their opening, is displaced in order to transfer an amplification reaction from the at least one microcavity and/or to combine several amplification reactions of different microcavities. Displacement may occur, for example, with water, an elution buffer, or a detergent-containing buffer. The amplification reactions can be transferred to a microfluidically connected system via the opening and the individual amplification reactions can be combined if necessary. In the context of the present invention, an amplification reaction is understood to mean the reaction solution located in the reaction compartment after amplification.

In an alternative advantageous embodiment for transferring an amplification reaction from the at least one microcavity and/or for merging a plurality of amplification reactions of different microcavities, the at least one microcavity has a further opening in the wall, in particular directed downwardly. Such a further opening is closed, for example, through an actuatable membrane during the reaction process. After the reaction has been completed, the further opening is opened and the actuatable membrane is removed in order to transfer the amplification reaction, in particular downwards, into a microfluidically connected system and, if necessary, to merge the individual amplification reactions.

For example, the microfluidically connected system is a microfluidically connected reaction chamber having a volume of, for example, 20 μl. Here, for example, the target amplifiers may be further enriched and purified. For further enrichment, the biomarker target amplification agents are re-amplified with the universal sequence as needed and read out by, for example, NGS. Based on the cell bar code sequence queried during selection, the biomarkers can be clearly assigned to the individual cells.

In one advantageous embodiment, the purification of the biomarker target amplifiers is carried out by means of capture molecules, in particular by means of capture molecules immobilized to magnetic beads. For example, the capture molecules, in particular streptavidin, bind to the selection marker of the SFP and SRP, in particular biotin. For this purpose, for example, corresponding binding conditions for the capture molecules and the biomarker target amplifiers comprising the selection markers are prepared and a “bind-wash-elute” method, which can be carried out according to known protocols, is applied.

Alternatively, instead of the classical elution, a restriction enzyme may also release, for example, the target amplifiers, for example from the capture molecule. A precondition for this is that corresponding interface sequences are built into the universal sequence in the SFP and SRP.

Here, in one embodiment of the invention, it is advantageous if the protelomerase TelN or an enzyme mutant thereof is used in an elution of the biomarker target amplifiers from the capture molecule, which covalently bonds the two single-stranded molecules of the DNA double helix (sense and antisense strand) at the interface, thereby leaving a hairpin structure. Such a hairpin structure is required, for example, for the production of NGS libraries, for example for the sequencing library of the sequencing technology of Pacific Biosciences. Advantageously, no further steps for adapter ligation need be carried out.

Furthermore, for example, further enrichment of the biomarker target amplifiers may be performed by isothermal Rolling Circle Amplification (RCA) without the need for additional sample preparation steps to be performed for amplification.

In one advantageous embodiment, the cells are circulating tumor cells (CTCs). For example, the molecular genetic single cell analysis of these CTCs is a highly sensitive way of collecting information about the course of therapy (prognostic) on the one hand cancer and about therapy adaptation (diagnostic) and, on the other, for example when monitoring the therapy of cancer patients. Here, the rare CTCs (for example 10¹-10² per mL of blood; individually or a few as a pool) must be isolated from a high background of ‘healthy’ blood cells (>10⁶ leukocytes, >10⁹ erythrocytes per mL of blood). According to conventional methods, the genetic material of these individual isolated cells must be pre-amplified in order to generate sufficient input for downstream analyses.

The method according to the invention eliminates process steps for pre-amplifying the DNA and/or RNA of the CTCs, as well as previous steps for merging multiple pre-sorted cells, which significantly shortens the process time, requires fewer work steps, requires fewer reagents and thus also results in lower costs.

Cells employed in the method according to the invention may be previously stained cells, e.g., EpCAM-positive CTCs. These carry the surface antigen EpCAM (epithelial cell adhesion molecule). However, all cells may also be considered without prior staining, for example all single cells released from the cell assembly of a tissue.

Alternatively, the cells may also be microbial cells or those of protozoa.

The object of the invention is furthermore a control unit configured to perform and/or control the steps of the method according to the invention in corresponding units, in particular in a microfluidic cartridge.

A further object of the invention is a computer program configured to perform and/or control the steps of the microfluidic method as well as a machine readable storage medium on which the computer program is stored.

### BRIEF DESCRIPTION OF THE DRAWINGS

Embodiments of the present invention are illustrated in the drawings and further explained in the subsequent description thereof. The figure shows:

FIG. 1: the schematic representation of one embodiment of a method according to the invention for combined cell-specific labeling and enrichment of biomarkers in a reaction compartment, and

FIG. 2: the schematic representation of a biomarker-specific forward or reverse primer.

### EMBODIMENTS OF THE INVENTION

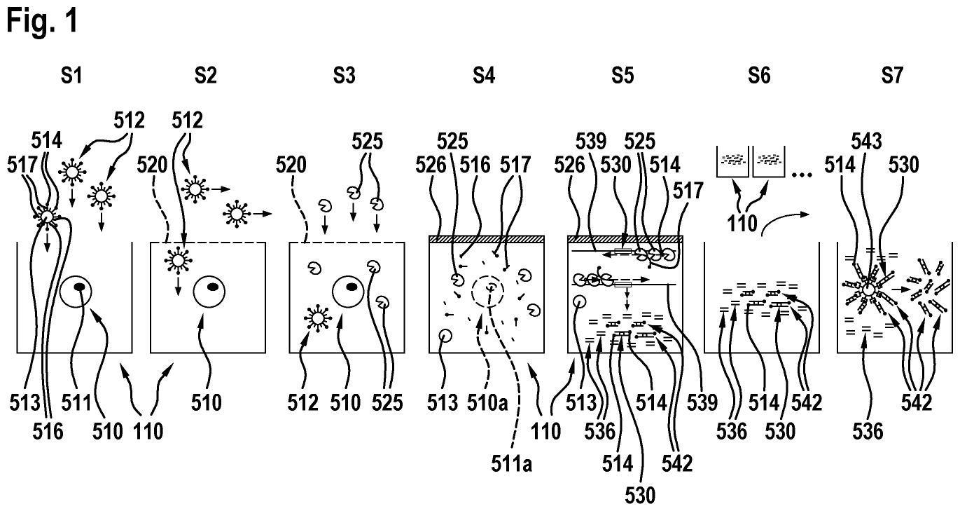

In FIG. 1, the method according to the invention for combined cell-specific labeling and enrichment of biomarkers in a reaction compartment is shown in an exemplary embodiment, which is described using an isothermal ssMDA.

A device with which the method according to the invention can be performed is described in the yet unpublished German application with file number 102022203848.7. This device a carrier substrate for receiving a sample liquid. The carrier substrate comprises at least one microcavity 110 and further comprises at least one electrode arranged at or in the microcavity 110 to generate an electrical field configured to trap a cell 510, in particular a nucleated cell 510, and/or a primer bead 512 and/or a further particle in the microcavity 110.

FIG. 1 of the present invention shows only one microcavity 110 of this device.

In a step 0 not shown in FIG. 1, which precedes this step, a first sample liquid with cells 510 is introduced into the microfluidic device or placed on the carrier substrate. The cells 510 sediment in the direction of the microcavities. Once a cell 510 approaches the microcavity 110, the DEP cage is transitioned to an open state or turned off so that this cell 510 can enter the microcavity. Once cell 510 has entered, the DEP cage is closed again so that no further cell can enter it and a maximum of one cell is contained per microcavity. If all cells are sedimented and, for example, all other microcavities are also loaded with one cell each, the sample liquid is rinsed away. The DEP cage remains closed so that the cell 510 is held until further notice.

In a step S1 of FIG. 1, a cell 510 having a nucleus 511, herein by way of example a CTC, is depicted in the microcavity 110. A second sample liquid with primer beads 512 is now introduced into the microfluidic device or added to the carrier substrate. Here, for example, the first sample liquid located above the carrier substrate, comprising the cells 510, is displaced.

The primer beads 512 comprise, for example, a particle 513 made of polystyrene, with different primer populations reversibly coupled thereto, herein exemplary biomarker-specific forward primers (SFP) 517 and biomarker-specific reverse primers (SRP) 517 with a selection marker 514 and random primer (RP) 516. In FIG. 1, the SFP 517 and SRP 517 are visually indistinguishable from one another, but it is noted that they are different populations of primers.

In step S2, the DEP cage is transferred in an open or turned off state so that a primer bead 512 can sediment into the microcavity 110

Once a primer bead 512 has sedimented into the microcavity 110, the DEP cage is transitioned to a closed state by a voltage change and the primer bead 512 is trapped with the cell 510 in the same. In this state, no further primer beads 512 can enter the microcavity 110 such that it is ensured that there is only a single primer bead 512 in the microcavity 110 in addition to the single cell 510. The dashed line with reference number 520 is intended to pictorially illustrate that the DEP cage is closed after loading with the primer bead 512.

In an alternative embodiment not shown in FIG. 1, the microcavity 110 may also be first loaded with a primer bead 512 and then with a cell 510.

In a further alternative embodiment not shown in FIG. 1, the primer beads may also only carry the SFP and SRP, and the RP is introduced in step S3 via the amplification mixture.

In a step S3, an amplification mixture is subsequently introduced into the microfluidic device or added to the carrier substrate. Here, either the second liquid with the unused primer beads 512 is first removed from the carrier substrate and the amplification mixture is subsequently added to the carrier substrate, or the liquid with the primer beads 512 is directly mixed with the amplification mixture on the carrier substrate. The amplification mixture comprises a buffer having an SD polymerase 525 and deoxyribonucleoside triphosphates (dNTPs), and optionally a reverse transcriptase and/or nicking enzyme. In FIG. 1, only the polymerase 525 is shown for ease of understanding.

In step S3, the diffusion-based distribution of the constituents or polymerase 525 of the amplification mixture into the microcavity 110 is illustrated. Here, the DEP cage is closed, which is illustrated by the dashed line with the reference numeral 520, since the components of the amplification mixture do not represent entities with dielectric properties and can therefore pass through the DEP cage in a diffusion-based manner even in the closed state.

The microcavity 110 now contains the cell 510, primer bead 512 and SD polymerase 525, as well as other constituents of the amplification mixture.

In a step S4, the amplification mixture is displaced above the at least one microcavity 110 by a non-aqueous phase 526, for example by an oil phase or air, such that the microcavity 110 is closed in the area of its opening by the non-aqueous phase 526 and thus represents a closed reaction compartment, in which the cell 510 and the primer bead 512 are enclosed. Conditions are now induced which lead to lysis of the cell 510 and release of the primers 516, 517 from the particle 513.

In S4, a lysed cell 510a is shown, from which the cell constituents, for example RNAs 539, have become free, as well as a lysed nucleus 511a, which has released the DNA 539 located therein. For example, such conditions are generated by raising the temperature to, for example, above 70 °C for, for example, more than one minute and/or by electroporation and/or by ultrasound.

In the temperature-induced lysis of the cell 510, denaturation of the DNA double strands and/or the RNA secondary structures and/or tertiary structures occurs simultaneously, particularly preferably at temperatures between 72 °C and 80 °C, for example for more than one minute.

In a further step S5 the amplification reaction is carried out by means of ssMDA. The random primers 516 bind non-directionally to regions of the DNA 539 and/or RNA 539 such that it can be used to perform isothermal strand-displacement-based amplification of the total DNA 539 and/or RNA 539 using polymerases 525. This results in total DNA amplifiers 539 and/or total RNA amplifiers 539. In addition, the specific forward primers 517 and the specific reverse primers 517 bind flanking upstream and flanking downstream of the biomarker targeting regions 530 of the DNA 539 and/or RNA 539, such that amplification of the biomarker target region 530 takes place, which is labeled in a cell-specific manner by the selection marker 514, for example biotin. For amplification, the polymerases 525 bind to the SFP and SRP, which provide the SD polymerases 525 with the starting point for amplification. This results in the creation of biomarker target amplifiers 542.

For example, the SFP and SRP carry additional nicking sequences at the 5′ end that extend the universal sequence. Upon prior addition of a corresponding nicking enzyme with the amplification mixture in step S3, an additional, disproportionate enhancement of the biomarker target amplification may be induced here, in which the biomarker target amplifiers 542 continue to carry the selection markers 514.

For example, the amplification of the RNA may be additionally enhanced by reverse transcriptase.

In a step S6, it is indicated that in order to merge multiple amplification reactions from different microcavities 110, the non-aqueous phase 526 above the microcavities 110, which had closed them in the area of their opening, is displaced. Displacement may occur, for example, with an elution buffer or a detergent-containing buffer. The amplification reactions can be transferred to a microfluidically connected system, for example a microfluidically connected reaction chamber, via the opening and the individual amplification reactions can be combined if necessary.

Here, for example, the target amplifiers 542 may be further enriched and purified.

In a step S7, the purification of the amplified biomarker target amplifiers 542 is carried out using capture molecules 543, which, for example, include magnetic particles (beads). The capture molecules 543, for example streptavidin, bind to the selection marker 514 of the SFP 517 and SRP 517, in particular biotin. Thus, the biomarker target amplifiers 542 may be separated from the remaining reaction constituents, such as total DNA amplifiers 539 and/or total RNA amplifiers 539. Subsequently, elution of the biomarker target amplifiers 542 from the capture molecules 543 is performed, for example by classical elution methods or by a restriction enzyme.

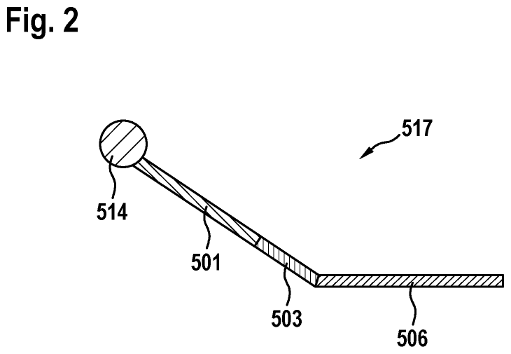

FIG. 2 shows an example of a biomarker-specific forward primer (SFP) 517 or a biomarker-specific reverse primer (SRP) 517.

The SFP or SRP comprises, starting at the 5′ end, a selection marker 514 coupled to a universal sequence 501, followed by a unique cell bar code sequence 503 and a specific biomarker target sequence 506.

By means of the specific biomarker target sequence 506, the SFP 517 attaches to the DNA 539 and/or RNA 539 in a flanking forward-directed manner up to 3 kb upstream of the biomarker target region 530 and the SRP 517 attaches to the DNA 539 and/or RNA 539 in a flanking backward-directed manner up to 3 kb downstream of the biomarker target region 530.

The unique cell bar code sequence 503 is clonally contained in each of the SFP 517 and SRP 517 of a primer bead 512, for example having a length of 8 or more bases.

The selection marker 514 is, for example, biotin at the 5′ end of the SFP 517 or SRP 517.

By means of the selection marker 514, subsequent positive selection of the biomarker target amplifier 542 is possible. For example, the universal sequence 501 is needed for subsequent amplification of a sequencing library or known sequence having a function of choice, for example a restriction interface, in particular for the release of the biomarker target amplifier 542, for example from a capture molecule 543.

Optionally and not shown in FIG. 2, the SFP 517 or SRP 517 has a unique molecular identifier (UMI) in the form of a randomized sequence. For example, the UMI may be located between the universal sequence 501 and the cell bar code sequence 503 or between the cell bar code sequence 503 and the specific biomarker target sequence 506. In particular, the UMI has a length of 6 or more bases.

Further optionally and not shown in FIG. 2, the SFP 517 or SRP 517 can be extended by a nicking sequence at the 5′ end of the universal sequence 501. The selection marker is thus no longer located at the end of the SFP or SRP but internally at the 5′ end of the universal sequence.

1. A microfluidic method for combined cell-specific labeling and enrichment of biomarkers in a reaction compartment, comprising
a) providing a microfluidic device comprising at least one carrier substrate for receiving a sample liquid, the at least one carrier substrate comprises at least one microcavity, and further comprising at least one electrode arranged at or in the at least one microcavity to generate an electrical field configured to trap a cell and/or a primer bead in the at least one microcavity;
b) loading the at least one microcavity of the microfluidic device with the cell and trapping the cell;
c) loading the at least one microcavity of the microfluidic device with the primer bead comprising at least one primer population coupled to the primer bead and trapping the primer bead wherein steps b) and c) can also be carried out in reverse order;
d) introducing an amplification mixture into the at least one microcavity;
e) displacing the amplification mixture above the at least one microcavity by a non-aqueous phase, so that the at least one microcavity represents a closed reaction compartment;
f) inducing cell lysing and primers releasing conditions; and
g) binding the at least one primer population to biomarker regions of DNA and/or RNA of the lysed cell for cell-specific labeling and amplification of a biomarker target region.

2. The microfluidic method according to claim 1, further comprising:
reversibly coupling different populations of primers to the primer bead, which is a polystyrene primer bead,
wherein the primers include biomarker-specific forward primers, biomarker-specific reverse primers, and/or random primers.

3. The microfluidic method according to claim 2, wherein the biomarker-specific forward primers and the biomarker-specific reverse primers each comprise a selection marker coupled to a universal sequence, a unique cell bar code sequence, and correspondingly a specific forward or reverse target sequence for the biomarker target region.

4. The microfluidic method according to claim 3, wherein multiple biomarker-specific forward primers and biomarker-specific reverse primers having different flanking specific forward or reverse target sequences are used for each of biomarker target region.

5. The microfluidic method according to claim 2, wherein the biomarker-specific forward primers and the biomarker-specific reverse primers further comprise a unique molecular identifier configured as a randomized sequence.

6. The microfluidic method according to claim 3, wherein the primer bead is configured such that the biomarker-specific forward primers and the biomarker-specific reverse primers are bonded to the primer bead via the selection marker or via a reversible compound.

7. The microfluidic method according to claim 3, wherein the universal sequence of the biomarker-specific forward primers and the biomarker-specific reverse primers is selected so that a direct attack surface is introduced for further amplification.

8. The microfluidic method according to claim 3, wherein the universal sequence of the biomarker-specific forward primers and the biomarker-specific reverse primers comprises a single-strand break sequence via which a single-strand break is inducible by a single-strand break-inducing enzyme and/or wherein the selection marker is coupled to an internal, non-terminal base of the universal sequence.

9. The microfluidic method according to claim 1, wherein the amplification mixture is cell lysis-supportive and/or comprises a single strand break-inducing enzyme.

10. The microfluidic method according to claim 1, wherein the amplification is an isothermal amplification.

11. The microfluidic method according to claim 1, wherein for transferring an amplification reaction from the at least one microcavity and/or for merging a plurality of amplification reactions of different microcavities, the non-aqueous phase is displaced or the at least one microcavity has a further opening in a wall.

12. The microfluidic method according to claim 1, wherein purification of biomarker target amplifiers occurs by capture molecules immobilized to magnetic beads.

13. The microfluidic method according to claim 12, wherein a protelomerase TelN or an enzyme mutant thereof is used in an elution of the biomarker target amplifiers from the capture molecules, which covalently bonds the two single strand molecules of DNA double helix at an interface and leaves behind a hairpin structure, for performing isothermal rolling circle amplification for further enrichment of the biomarker target amplifiers.

14. The microfluidic method according to claim 1, wherein the cells are circulating tumor cells.

15. A control unit configured to execute and/or control steps of a method according to claim 1.

16. The method according to claim 1, wherein a computer program is configured to execute and/or control the steps of the microfluidic method.

17. A non-transitory machine-readable storage medium on which the computer program according to claim 16 is stored.
